(19) United States
(12) Patent Application Publication
(10) Pub. No.: US 2021/0113151 A1
(43) Pub. Date: Apr. 22, 2021

(54) Device and Method for Examining Metabolic Autoregulation

(71) Applicant: Imedos Systems GmbH, Jena (DE)

(72) Inventors: Walthard VILSER, Rudolstadt (DE); Benedikt KRAUSS, Jena (DE); Thomas RIEMER, Jena (DE)

(21) Appl. No.: 17/042,827

(22) PCT Filed: Jul. 12, 2018
(86) PCT No.: PCT/DE2018/100639

§ 371 Date: Sep. 28, 2020

(30) Foreign Application Priority Data
Mar. 29, 2018 (DE) 10 2018 107 621.5

(51) Int. Cl. A61B 5/00; A61B 3/12; A61B 3/14; A61B 3/16; A61B 3/18; A61B 3/00; G16H 30/40; G16H 40/63; G16H 50/30; H04N 9/04

(52) U.S. Cl. CPC A61B 5/4866; A61B 3/1233; A61B 3/1241; A61B 3/14; A61B 3/16; A61B 3/18; H04N 9/0455; A61B 5/725; A61B 5/7278; G16H 30/40; G16H 40/63; G16H 50/30; A61B 3/0025

(57) ABSTRACT

The invention relates to a method and to a device suitable therefor, for examining the metabolic autoregulation of the retinal vessels in a patient's eye. Over a baseline phase (BP), a stimulation phase (SP), in which the individual retinal perfusion pressure in the eye is decreased in a normalized manner by the targeted application of a stimulation pressure to the eye, and a posterior phase (NP), a video sequence of images of the retina is captured, from which images signals describing the local vascular perfusion can be derived, from which signals metabolic vascular reactions are derived and recorded in order to examine the vascular reactions of the large retinal vessels e.g. on the basis of vessel diameter signals (D(t,x,y) and in order to examine the capillary vessels e.g. on the basis of spectrally normalized quotient signals.

### RELATED APPLICATIONS

This Application is a U.S. National Stage Under 35 USC § 371 of International Application PCT/DE2018/100639, filed on Jul. 12, 2018, which in turn claims priority to German Patent Application DE 10 2018 107 621.5, filed Mar. 29, 2018, both of which are incorporated herein by reference in their entirety.

### FIELD OF THE INVENTION

The invention relates to a device and a method by which the metabolic autoregulation of the retinal vessel system in the eye of a patient can be examined by artificially applying a stimulation pressure SD. This makes the device and the method, as a functional examination, suitable for use in the causal diagnosis of retinal blood flow disorders, for course observation and monitoring therapeutic effects, but above all for the preventive diagnosis of glaucoma and other retinal blood flow disorders.

### BACKGROUND OF THE INVENTION

Autoregulation refers to various autoregulatory mechanisms with different functions and dynamics. Metabolic autoregulation is understood to mean that the retinal vessels react to deficiencies when the metabolism of the retina is no longer adequately supplied with oxygen and nutrients or is disturbed. This is the case, for example, when the retinal perfusion pressure rPP of the retina drops. In this case, autoregulation serves to compensate for the drop in pressure by dilating the retinal vessels.

Various scientific results and technical proposals use methods to measure blood flow or blood velocity or to detect capillary occlusions (fluorescence angiography, OCT angiography). However, the imaging systems known so far can only show morphological damage but not functional disorders. Thus, the prior art in current morphological imaging has significant disadvantages and will not be considered further below.

Methods for examining functional autoregulatory mechanisms based on vessel diameters are described in the literature. The examination of metabolic autoregulation based on the measurement of vessel diameters is described in the work of Nagel et al (Nagel, E.; Vilser, W.: “Autoregulative behavior of retinal arteries and veins during changes of perfusion pressure: a clinical study.” Graefe's Arch Clin Exp Ophthalmol (2004) 242: 13-17), which comes closest to the inventive solution. Using the suction cup method according to Ulrich, the intraocular pressure IOP is artificially increased to a constant value. The required stimulation pressure SD (negative SD values in the suction cup) is then kept constant over a stimulation period T (e.g. 90 s) before being relieved to zero. The Retinal Vessel Analyzer from Imedos was used to measure vessel diameters of selected large retinal arteries and veins, vessel segment by vessel segment along selected vessel segments over a total time in three phases. In the first phase (baseline phase BP), vessel diameters of the large retinal vessels are examined, uninfluenced by changes in IOP or rPP, i.e. without stimulation (provocation). In a second phase (stimulation phase SP), the retinal perfusion pressure rPP is decreased or the intraocular pressure IOP is rapidly increased by a predetermined change intraocular pressure value dIOPₛ and kept constant for the stimulation period T. In the third phase (posterior phase NP) the dIOPₛ is quickly relieved to zero. The vessel diameters averaged over the vessel sections were recorded over the total time as vessel diameter signals D(t,x,y). The vessel diameter signals D(t,x,y) show significant vascular reactions in the patient's mean values in phases SP and NP, which reactions describe metabolic autoregulation. The baseline phase BP serves to determine reference values to which the vascular reactions are standardized on a percentage basis.

A major disadvantage of the aforementioned prior art is that the metabolic vascular reaction of the capillaries cannot be examined Since it must be assumed that the vascular regions of the arterial, venous and capillary vessels exhibit different vascular reactions and that a significant part of the mass transfer takes place in the capillaries, knowledge of capillary metabolic autoregulation is of particular interest or at least necessary in order to generally understand and clinically classify metabolic autoregulation.

Further disadvantages of the examination described in the aforementioned prior art consist in the choice of the suction cup method according to Ulrich. The method already increases the IOP uncontrollably when the suction cup is attached to the eye, causes lesions with bruising in the patient's eye due to the suction pressure and is tolerable but unpleasant for the patient. The suction effect also deforms the eyeball and leads to astigmatism at a very early stage, resulting in measurement errors when measuring vascular reactions. In addition, there is a very large spread of vascular reactions. The main causes are tonographic effects and the uncertain relationships used between suction pressure and change in intraocular pressure dIOP, which were determined as a mean relationship from many different eyes and are used in the suction cup method to calculate the IOP values for the examinations. Additional tonometer measurements showed that the tonographic effect is negligible compared to the large uncertainty of the calibration relationship of the OODG device according to Ulrich between dIOP and suction pressure.

### SUMMARY OF THE INVENTION

It is the object of the invention to find a method by which the capillary metabolic vascular reactions can be examined in parallel with the vascular reactions of the large vessels. Moreover, advantageously, the reproducibility and the individual significance of the examination results should be considerably improved. Advantageously, the examination should be made more comfortable for the patient.

It is also the object of the invention to provide a device suitable to carry out the method.

The essence of the invention consists in capturing the capillary vessels in parallel with the measurement of the vessel diameters of the large retinal vessels by spectrally normalized quotient signals or other local signals describing the capillaries, such as the moving blood cell density (also called capillary density), the capillary flow or capillary velocity or their plasma or blood cell movement, and thus recording their vascular reactions.

As a major uncertainty and source of error in the clinical conclusion concerning the examination of autoregulation, as obtained with the previously described method, it was recognized that for the examination-based reduction of the perfusion pressure rPP, only the resting intraocular pressure IOP₀, regardless of the retinal venous pressure outside the eyeball RVP, was considered as the initial value.

It is essential to the invention that the influence of increased retinal venous pressure outside the eyeball RVP on the examination of vascular reaction and autoregulation be eliminated or that the examination be standardized with respect to a current individual retinal perfusion pressure rPP.

Also, advantageously, the strong scatter of the correlation between the IOP and the stimulation pressure SD is considerably reduced by direct measurement of the IOP or by a correlation between the IOP and SD determined individually for the eye in question. Thus, the mean and highly scattering relationship between the IOP and SD, as used in the aforementioned prior art, is replaced by a precise individually determined relationship.

The retinal perfusion pressure rPP is calculated from the difference between the retinal arterial blood pressure rPa and the retinal venous blood pressure rPv inside the eyeball. This retinal venous blood pressure rPv within the eyeball usually corresponds to the natural intraocular pressure IOP (equal to the resting intraocular pressure IOP₀) in healthy eyes. The IOP₀ is then greater than the retinal venous pressure RVP prevailing outside the eye. In this case, a so-called spontaneous venous collapse can be seen on the papilla (optic nerve head). The retinal perfusion pressure rPP is then calculated as follows:

rPP=rPa−rPv if IOP₀>RVP, then rPv=IOP and rPP=rPa−IOP

If there is no venous collapse, this is an indication that the RVP is greater than the IOP₀, and the RVP then determines the retinal perfusion pressure rPP according to the following formula:

rPP=rPa−rPv if RVP>IO₀, then rPv=RVP and rPP=rPa−RVP

Consequently, the retinal perfusion pressure rPP is only determined by the IOP₀ of the eye if the latter is greater than the retinal venous pressure outside the eyeball RVP. However, in pathological cases, especially in glaucoma, the RVP is often higher than the IOP₀, and thus the RVP determines the retinal perfusion pressure rPP, as can be seen from the above formulas.

Based on these findings, metabolic autoregulation is examined during a standardized reduction of the retinal perfusion pressure rPP. This reduction is related to the actual rPP and is based on an IOP value at which spontaneous venous collapse is detected.

If spontaneous venous collapse is already detected without an increase in the IOP, then the initial IOP value, which is the resting intraocular pressure value IOP0 when a pressure applicator is applied without pressure, is increased by a predetermined change intraocular pressure value dIOPs to a stimulation intraocular pressure value IOPs. In this case, the retinal stimulation perfusion pressure rPP during the stimulation phase SP is:

rPPₛ=rPP−dIOPₛ=rPa−IOP₀−dIOP, (for IOP₀>RV) (formula 1)

The prior art examinations were also carried out under these conditions.

According to the invention, taking into account a retinal venous pressure outside of the eyeball RVP which is increased over IOP₀, i.e. RVP>IOP₀, the following calculation formula results for rPP:

rPPₛ=rPP−dIOPₛ=rPa−RVP−dIOPₛ=rPa−IOP₀−dIOP_{RVP}−dIOPₛ (for IOP₀<RVP) (formula 2)

where the value dIOP_{RVP} is the value by which the IOP₀ must be increased to trigger the spontaneous venous collapse and measure the RVP.

The effects of neglecting an RVP when it is greater than the resting intraocular pressure IOP₀ can be seen in formula 2. To achieve an equal reduction of the retinal perfusion pressure rPP by the predetermined dIOPₛ when the RVP is increased (RVP>IOP₀), it is not required to increase the IOP by dIOPₛ compared to IOP₀ but compared to dIOPₛ+dIOP_{RVP}. The value dIOP_{RVP} is exactly the value by which RVP is greater than IOP₀.

Clinically this means that in the case of the prior art, increasing the IOP by dIOPₛ according to formula 1 is correct for IOP₀>RVP, but is wrong in the other case, because in the extreme case the retinal perfusion pressure rPP was not reduced at all, so that in this case no vascular reaction is to be expected. For the prior art, it can therefore be assumed that rPP was not standardized in the studies but reduced to a very different extent or even not reduced at all in some cases. This results in a wide spread of vascular reactions, depending on the composition of the patient group and their RVP or IOP values.

According to the invention, the RVP is taken into account when calculating the stimulation pressure value SDₛ associated with the IOP, thus eliminating a significant error influence, especially in pathological cases.

With respect to a device for examining the metabolic autoregulation of the retinal vessels in a patient's eye, said device comprising a unit for generating and applying a stimulation pressure, which acts on the eye, and an imaging unit, the object of the invention is achieved in that the imaging unit is a modified retinal camera with a digital image sensor, which generates a video sequence of images of the retina, to each of which images two color channels are assigned, and a unit for generating spectrally normalized quotient signals is present which derives quotient signals from intensity signals of the color channels, from which quotient signals it is possible to infer the vascular reaction and thus the metabolic autoregulation of the capillaries of the retinal vessels. Alternatively, the object is achieved, with respect to a device, in that the imaging unit is based on laser scanning technology or optical coherence tomography, which generates a video sequence of images of the retina, from which images signals can be derived to describe local vessel diameters, local blood velocities, local blood flows or local capillary densities of the capillaries or/and the large vessels. Further, the object is achieved, with respect to a device, in that a tonometer is present to measure an intraocular pressure IOP in the eye, said intraocular pressure IOP changing as a function of a stimulation pressure applied by the unit for generating and applying a stimulation pressure, and in that the unit for generating and applying a stimulation pressure includes a sensor for measuring the stimulation pressure in order to be able to assign a stimulation pressure value to a respective measured intraocular pressure value IOP and to each image of the video sequence.

The object of the invention can be achieved on the basis of different imaging units, wherein respective signals of the large vessels and capillaries are formed which describe the local vessel diameter or capillary density, blood cell or blood plasma velocity or blood flow or otherwise the vascular perfusion.

Advantageously, the unit for generating and applying a stimulation pressure comprises a pressure applicator, which can be attached to the patient's head, fixed with respect to the eye, outside the cornea and outside a light path of the imaging unit, in pressure-free, planar contact with the eye.

Further advantageously, the imaging unit is a spectrally modified retinal camera having in its illumination beam path a double band-pass filter with a spectral range in red light and a spectral range in green light.

With respect to a method for examining the metabolic autoregulation of the retinal vessels in a patient's eye, wherein a video sequence of images of the retinal vessels is recorded during a baseline phase which does not affect the eye, a stimulation phase in which an intraocular pressure IOP is increased by a predetermined change intraocular pressure value dIOP, by applying and increasing a stimulation pressure acting on the eye and is maintained at a stimulation intraocular pressure value IOPₛ for a stimulation period, and a posterior phase which does not affect the eye, and wherein signals which describe the local perfusion of vessels (vascular perfusion) are derived from the images of the video sequence, the object of the invention is further achieved in that the increase by the predetermined change intraocular pressure value dIOPₛ is carried out starting from a measured resting intraocular pressure value IOP₀ if, during the baseline phase, measurement criteria for a spontaneous venous collapse at the optic nerve head are determined on the retina, in that the increase is carried out starting from an increased intraocular pressure value IOP_{RVP}, if during an increase in intraocular pressure IOP measurement criteria for spontaneous venous collapse at the optic nerve head are determined on the retina when the increased intraocular pressure value IOP_{RVP} is reached, and in that the increase is carried out on the basis of the measured resting intraocular pressure value IOP₀ if no spontaneous venous collapse is detected, not even during the increase in intraocular pressure IOP at the optic nerve head on the retina.

Preferably, the signals describing the vascular reaction of the retinal vessels for arterial and venous vessels are vessel diameter signals and the signals describing capillary vessels are spectrally normalized quotient signals which are acquired and recorded in parallel.

At least a second intraocular pressure value IOP is preferably measured after the measurement of the resting intraocular pressure value IOP₀ under the action of a stimulation pressure SD, and an individual relationship is determined for the relevant eye between the intraocular pressure values IOP and the respectively associated stimulation pressure values SD in order to set the stimulation intraocular pressure value IOPₛ via an individually associated stimulation pressure value SDₛ.

Moreover, it is preferable, when the stimulation phase starts, to increase the stimulation pressure SD by at least 1 mmHg per second in order to rapidly increase the resting intraocular pressure value IOP₀ by the predetermined change intraocular pressure value dIOPₛ to the stimulation intraocular pressure value IOPₛ.

Preferably, after the stimulation period has elapsed, the stimulation phase is terminated and the stimulation pressure SD is suddenly reduced to zero.

### BRIEF DESCRIPTION OF THE DRAWINGS

The invention will be explained in more detail below on the basis of drawings with reference to an exemplary embodiment of a device and of a method using a modified conventional retinal camera. In the drawings:

FIG. 1 shows a block diagram of a device according to the invention;

FIG. 2 shows an embodiment of a unit for generating and applying a stimulation pressure;

FIG. 3A shows a time sequence for the examination when the retinal perfusion pressure rPP results from the resting intraocular pressure value IOP₀;

FIG. 3B shows a time sequence for the examination when the retinal perfusion pressure rPP results from an increased intraocular pressure value IOP_{RVP}, which corresponds to an RVP greater than the resting intraocular pressure IOP₀;

FIG. 4A shows a time sequence of a vessel diameter signal for an artery and a vein during the three examination phases, and

FIG. 4B shows a time sequence of a quotient signal during the three examination phases.

### DETAILED DESCRIPTION OF THE PREFERRED EMBODIMENTS

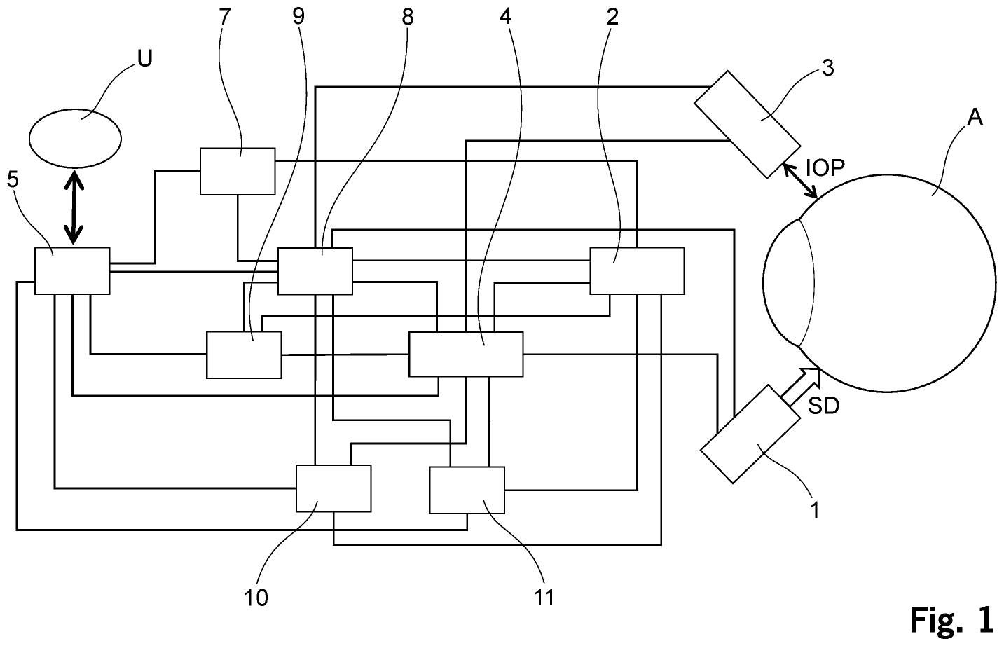

As shown in a block diagram in FIG. 1, a device according to the invention comprises at least one unit for generating and applying a stimulation pressure 1, an imaging unit 2 with a digital image sensor, a tonometer 3, a computing and control unit 4, an input and output unit 5, a results image storage unit 7, a data and image evaluation unit 8, a signal analysis unit 9, a unit for generating spectrally normalized quotient signals 10 and a unit for generating vessel diameter signals 11.

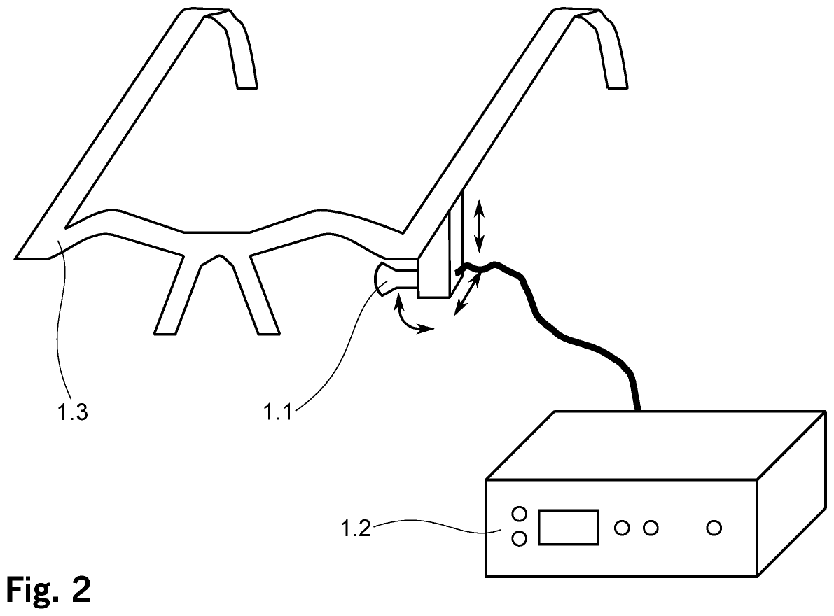

The unit for generating and applying a stimulation pressure 1 comprises, as schematically shown in FIG. 2, a pressure generating unit 1.2, a holder 1.3 and a pressure applicator 1.1, which is fixed to the patient's right and left eyes A laterally (temporally), in each case, via the holder 1.3, which preferably resembles a pair of glasses. The pressure applicator 1.1 can be placed in pressure-free, planar contact with the patient's eye A to be examined.

The pressure applicator 1.1 serves to apply a stimulation pressure SD onto the patient's eye A to be examined and is preferably a small pneumatic balloon, but could also be, for example, a die, a suction cup or an hydraulic system.

In contrast to pressure applicators known from prior art devices or methods, the embodiment of the pressure applicator 1.1 as a small pneumatic balloon has a number of advantages. For instance, there is a much lower risk of injury from sharp rims, which are produced on the edges of pressure applicators 1.1 made of metals, plastic materials or ceramics or other solid materials. Moreover, the soft surface of the balloon is much more pleasant for the patient during the examination. In addition, the uniform expansion of the balloon in all directions avoids transverse forces which may result in falsified measurement results.

Connected to the pressure applicator 1.1 is the pressure generating unit 1.2, by which the stimulation pressure SD can be generated, increased, decreased or kept constant.

In order to generate the stimulation pressure SD in a controlled manner, the unit for generating and applying a stimulation pressure 1 is connected to the computing and control unit 4. Depending on the pressure applicator 1.1 selected, the unit for generating and applying a stimulation pressure 1 may comprise, as the pressure generating unit 1.2, for example, a pump, a system consisting of a pneumatic cylinder and a piston or/and control electronics for linear drives. Advantageously, the pressure generating unit 1.2 is a pneumatic system consisting of a pneumatic cylinder and a piston which can be moved in the pneumatic cylinder by a linear drive. By moving the piston, the air contained in the pneumatic system is compressed or dilated, causing the pressure in the pressure applicator 1.1 either to increase or to decrease. The pressure generating unit 1.2 may advantageously comprise components for defined adjustment of the increase or decrease in stimulation pressure SD. Possible designs for this include, for example, systems consisting of various throttle valves and magnetic valves, or suitable control electronics which allow different speeds of adjustment of a linear drive.

The pressure generating unit 1.2 comprises a sensor for measuring the stimulation pressure SD. Depending on the design of the unit, pressure sensors, force sensors or distance sensors may be used, for example.

The pressure generating unit 1.2 advantageously also comprises a component enabling a sudden drop in the stimulation pressure SD. For this purpose, one or more magnetic valves may be used, for example, by which the system is suddenly vented in an emergency.

The holder 1.3 serves to couple the pressure applicator 1.1 directly to the patient's head and may be, for example, a pair of glasses, a head band or a bracket placed on the patient's head. Preferably, the holder 1.3 is implemented in the form of a pair of glasses. In order to improve the coupling of the pressure applicator 1.1 to the patient's head, the holder 1.3 is advantageously provided with a further component, such as a glasses lace, a rubber band or a mechanically adjustable fixing means.

In order to achieve individually adaptable positioning of the pressure applicator 1.1, in particular adjustability of the direction from which the pressure applicator 1.1 is adducted to the patient's eye A, the pressure applicator 1.1 is attached to the holder 1.3 on the patient's eye A in an individually adjustable manner, preferably via height adjustment, distance adjustment and angle adjustment.

The optical access (light path) to the retina for the imaging unit 2 must not be impeded and/or blocked by any of the components contained in the unit for generating and applying a stimulation pressure 1.

In this case, the tonometer 3 is advantageously a modified rebound tonometer. It is connected to the computing and control unit 4 and to the data and image evaluation unit 8 via signaling pathways. It is integrated into the device and fully automatically controlled by it. Via the connection to the computing and control unit 4, automatically performed measurements of the intraocular pressure IOP are triggered upon reaching previously defined measurement criteria. The obtained intraocular pressure values IOP are transmitted to the computing and control unit 4 and are synchronized there to a time signal for further processing. The intraocular pressure values IOP synchronized to the time signal are transmitted to the data and image evaluation unit 8 for storage and further processing.

The computing and control unit 4 has a signal connection to the unit for generating and applying a stimulation pressure 1, the imaging unit 2, the tonometer 3 and the input and output unit 5, the data and image evaluation unit 8, the signal analysis unit 9, the unit for generating spectrally normalized quotient signals 10 and the unit for generating vessel diameter signals 11. The function of the data and image evaluation unit 8, which is connected via signal lines to the computing and control unit 4, the results image storage unit 7, the input and output unit 5, the imaging unit 2, the unit for generating and applying a stimulation pressure 1, the signal analysis unit 9, the unit for generating spectrally normalized quotient signals 10 and the unit for generating vessel diameter signals 11, will be explained by describing the method.

The results image storage unit 7, which is connected via a signal connection to the imaging unit 2, the input and output unit 5, and the data and image evaluation unit 8, is used to store or temporarily store measured values or signals reflecting the vascular reactions in result images such as the mapping image.

Signals that are transmitted to the signal analysis unit 9 for analysis are, for example, vessel diameter signals D(t,x,y). They are formed as a time- and location-dependent vessel diameter change of individual vascular segments or the average vessel diameter averaged for a vessel section formed from several vascular segments in the unit for generating vessel diameter signals 11. Other such signals over a defined area of a pixel or group of pixels (measurement location) can be e.g. an averaged unnormalized brightness signal or/and an averaged spectrally normalized quotient signal Q(t,x,y).

The input and output unit 5 is used by an examiner U to input data and control commands and to represent and output the respective examination results. During the examination, the stimulation pressure values SD and the video sequences can be represented online on the monitor. The examiner U can observe the video sequences on a monitor belonging to the input and output unit 5 and have the finished examination results presented. The video sequences are preferably presented to the examiner U together with measurement results for tracking and monitoring the adjustment of the imaging unit 2 during the examination procedure.

For example, the imaging unit 2 is a spectrally modified retinal camera that generates video sequences of images with two color channels of the retina.

Advantageously, the imaging unit 2 has a dual band-pass filter in its illumination beam path, e.g. with one spectral range in the red light and one spectral range in the green light, and the spectral components are directed to separate areas or different chips of a digital image sensor, resulting in a red and a green image, each of which can be understood as one of the two color channels of an image.

Alternatively, the imaging unit 2 can have a digital image sensor in which each pixel is formed by at least two subpixels of different spectral sensitivity, e.g. red and green. Each of the subpixels of a pixel of the digital image sensor generates one of two color channels of the images of the video sequence. The unit for generating spectrally normalized quotient signals 10 forms quotients, pixel by pixel, from the red and green color intensity signals of the color channels of the images or of the subpixels which together form a pixel. The pixels in successive images must correspond to the same measurement location. This results in a spectrally normalized quotient image, wherein illumination-side differences are eliminated by spectral normalization. The red backscattered light, which substantially penetrates blood, serves as the reference wavelength in this case, with light in the green light range being strongly absorbed by blood and reflecting the blood volume in a retinal region. Thus, the spectrally normalized quotient signal Q(t,x,y) describes, regardless of illumination, the blood volume in a capillary region. The resulting quotient image sequence of the retina is stored in the unit for generating spectrally normalized quotient signals 10 and then transmitted to the signal analysis unit 9 as spectrally normalized quotient signal Q(t,x,y) in accordance with the method steps. Any other imaging device that can produce images with multiple color channels can also be used as imaging unit 2.

For the purposes of the invention, it is irrelevant how and over which wavelengths the spectral normalization takes place, provided that illumination-independent signals are produced which describe the blood volume in a tissue volume.

The signal analysis unit 9 is connected to the input and output unit 5, the data and image evaluation unit 8, the computing and control unit 4, and the imaging unit 2. Its mode of operation will be explained later by describing an exemplary embodiment of the method according to the invention.

The unit for generating spectrally normalized quotient signals 10 is connected to the input and output unit 5, the data and image evaluation unit 8 and the imaging unit 2. As already explained, the unit for generating spectrally normalized quotient signals 10 serves to eliminate the influence of the illumination intensity on the signals generated for the examination.

The unit for generating vessel diameter signals 11 is connected to the input and output unit 5, the data and image evaluation unit 8 and the imaging unit 2. The unit for generating vessel diameter signals 11 determines vessel diameters in at least one selected vessel, segment by segment along the vessel sections, as well as image by image in the preferably green color channel of the video sequence or optionally in the quotient image. The time sequence of the vessel diameters of the individual vascular segments is then used to generate vessel diameter signals D(t,x,y) which are fed to the signal analysis unit 9.

The device need not necessarily include both a unit for generating spectrally normalized quotient signals 10 and a unit for generating vessel diameter signals 11 nor need it necessarily generate spectrally normalized quotient images and spectrally normalized quotient signals Q(t,x,y) derived from them. The proposals presented in this exemplary embodiment represent advantageous embodiments.

As to performing a method according to the invention, explanations were already given in the description of the essence of the invention and in the description of the functioning of the device, which are to be attributed to the following description of the process sequence, even if not explicitly referred to in each case.

In preparation for the examination, the examiner U attaches the pressure applicator 1.1 to the patient's head such that the pressure applicator 1.1 slightly touches the patient's eye A in the temporal canthus, without applying pressure.

Then the examiner U adjusts the tonometer 3 to the eye A such that automatic tonometer measurements are possible in parallel with imaging by the modified retinal camera and an evaluable image of the retina, including the optic nerve head (ocular fundus), can be provided on the monitor of the input and output unit 5.

The video sequences of images provided by the imaging unit 2 are automatically examined for sufficient image quality. If the image quality is not sufficient, the examiner U is prompted via the input and output unit 5 to correct the image quality by adjusting the modified retinal camera forming the imaging unit 2.

Then the examiner U starts the examination process with a first phase, the so-called baseline phase BP.

The intraocular pressure IOP is measured automatically and the measured intraocular pressure value IOP, equal to the resting intraocular pressure value IOP₀, is stored as an initial value.

Subsequently or during this operation, a video sequence of images, to which two color channels are assigned, is generated using the modified retinal camera over the duration of three phases of the examination. In addition to vessel diameter signals D(t,x,y), spectrally normalized quotient signals Q(t,x,y) in particular are formed from these images to evaluate the metabolic autoregulation of capillaries.

In the baseline phase BP, all signals are formed without increasing the stimulation pressure SD. They serve as reference values for a later evaluation of the signals via the subsequent stimulation phase SP and the posterior phase NP.

Prior to this, the video sequences of retinal images generated by the imaging unit 2 are analyzed by the data and image evaluation unit 8 for image shifts or rotations between temporally successive images and corrected in such a way that a movement-corrected video sequence is generated in which identical retinal points overlap in the images. All signals are generated on the basis of this movement-corrected video sequence.

To examine arterial and venous vessels, vessel diameter signals D(t,x,y) are formed from the movement-corrected video sequence. For this purpose, the arterial and venous vessels in the area of the papilla (optic nerve head) are selected and their measurement location is stored. The unit for generating vessel diameter signals 11 then accesses the stored arterial and venous vessels and determines vessel diameters along the vessels, segment by segment and image by image, the values of which are assigned to the measurement location on the retina and the time or the respective image and stored, and forms a time- and location-dependent vessel diameter signal D(t,x,y). Individual pixels or pixel groups can be assigned to the measurement locations.

The data and image evaluation unit 8 forms green color intensity signals and red color intensity signals from the color channels of the images of the video sequence or intensity values of the assigned pixels or subpixels, respectively, and forms spectrally normalized quotient signals Q(t,x,y) whose time sequence is assigned to the pixels at the measurement location on the retina, to a time signal to which all measurement results are also synchronized in the form of measured values or signals, or to the images.

All signals formed are fed to the signal analysis unit 9 and recorded (stored) in parallel as time sequences.

The computing and control unit 4 assigns all current SD values to a time signal, which is set to ZERO with the first START signal for the examination and to which all original and derived video sequences, images, quotient images and signals are synchronized or temporally assigned.

During the baseline phase BP and the subsequent increase of the intraocular pressure IOP at the beginning of the stimulation phase SP, the signal analysis unit 9 monitors all signals with regard to the objective measurement criteria defined below:

The signals on the selected optic nerve head are monitored for the occurrence of a spontaneous venous collapse. The following objective measurement criteria are used:

The diameters of individual venous vascular segments on the optic nerve head begin to pulsate significantly more than in the past or more than most venous vascular segments on the optic nerve head. The threshold factor for the increase in pulsation amplitude, which determines when the measurement criterion has occurred, is set at 3, but can be adjusted differently based on experimental investigations.

The signals, i.e. the quotient signal Q(t,x,y) and/or red color intensity signal and/or green color intensity signal, from the area of the optic nerve head increase significantly in their pulse amplitude compared to the past or to the signals of neighboring pixels. The threshold factor for the increase in pulsation amplitude, which determines when the measuring criterion has occurred, is set at 3, but can be adjusted differently on the basis of experimental investigations or between the different signals.

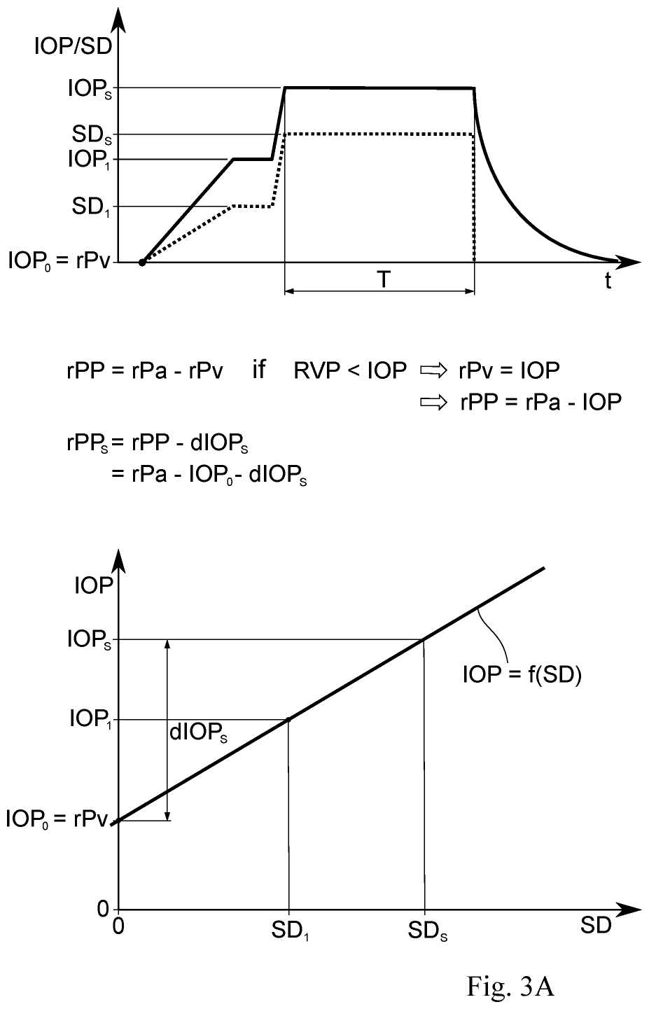

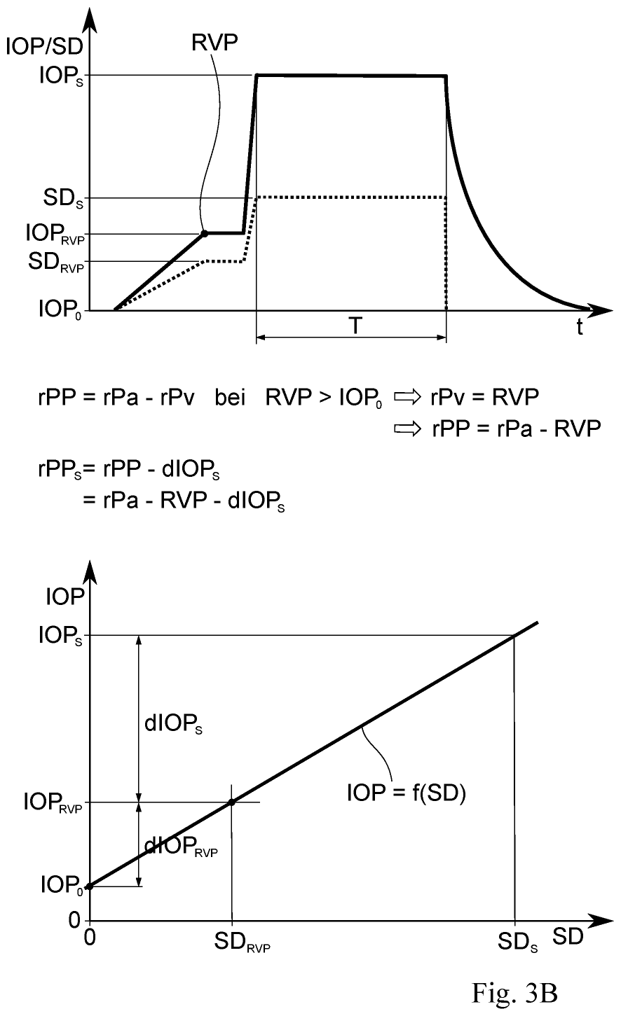

If the signal analysis unit 9 already detects during the baseline phase BP that at least one of the measurement criteria for venous collapse is met, see FIG. 3A, the resting intraocular pressure value IOP₀ is equated to the retinal venous blood pressure within the eyeball rPv. In this case, the resting intraocular pressure value IOP₀ determines the retinal perfusion pressure rPP and the above formula 1 is used, as in the prior art. A desired stimulation intraocular pressure value IOPₛ results from the addition of the resting intraocular pressure value IOP₀ and a predetermined change intraocular pressure value dIOPₛ for stimulation of the eye A.

IOPₛ=IOP₀+dIOP,

Not only in this case, but also if no spontaneous venous collapse has been detected, the stimulation phase SP is started via the computing and control unit 4. A START signal is fed to the unit for generating and applying a stimulation pressure 1 and the increase in stimulation pressure SD in the pressure applicator 1.1 is triggered. The stimulation pressure SD in the pressure applicator 1.1 should increase by at least 1 mmHg per second to rapidly increase the resting intraocular pressure value IOP₀ by the predetermined change intraocular pressure value dIOP, to the stimulation intraocular pressure value IOPₛ.

During the increase of the stimulation pressure values SD, at least one further intraocular pressure measurement is performed, indicated as IOP₁ in FIG. 3A, in order to determine the individual correlation of the causative stimulation pressure SD for eye A as a function of the achieved intraocular pressure IOP or of its change, SD=f(IOP), and to calculate a stimulation pressure value SDₛ associated with the stimulation intraocular pressure value IOPₛ, which must ultimately be reached by the unit for generating and applying a stimulation pressure 1 and kept constant over a stimulation period T. For increased accuracy, further IOP values can be determined during the SD increase, which are included in the calculation of the individual correlation SD=f(IOP).

In the absence of spontaneous venous collapse during the baseline phase BP, the signal analysis unit 9 continues its monitoring for the onset of spontaneous venous collapse during the SD increase, see FIG. 3B.

As soon as the signal analysis unit 9 detects spontaneous venous collapse on the optic nerve head during the SD increase, the increased intraocular pressure value IOP_{RVP} then associated with the retinal venous pressure outside the eyeball RVP is measured by the computing and control unit 4 and a specific change in intraocular pressure dIOP_{RVP} is determined from the difference with the resting intraocular pressure IOP₀, i.e. dIOP_{RVP}=IOP_{RVP}−IOP₀ or IOP₀+dIOP_{RVP}=RVP. According to the described formula 2 and the individual correlation SD=f(IOP), a new stimulation pressure value SD_{Snew}=f(dIOP_{RVP}) to be set is then calculated in order to increase the intraocular pressure IOP, equal to the RVP, to a newly calculated stimulation intraocular pressure value IOPₛ by changing it by the given change intraocular pressure value dIOPₛ.

If the venous collapse did not occur until the predetermined stimulation intraocular pressure value IOPₛ was reached or until the associated stimulation pressure value SDₛ was reached and consequently no intraocular pressure value IOP_{RVP} could be determined for the RVP, the stimulation intraocular pressure value IOPₛ is set for the case IOP>RVP (formula 1) and no new stimulation intraocular pressure value IOPₛ is calculated and generated by setting a new associated stimulation pressure value SDₛ.

After the stimulation period T has elapsed, the stimulation phase SP is terminated and the stimulation pressure SD is suddenly reduced to zero.

The recording of the signals is continued for a period of the posterior phase NP and then terminated.

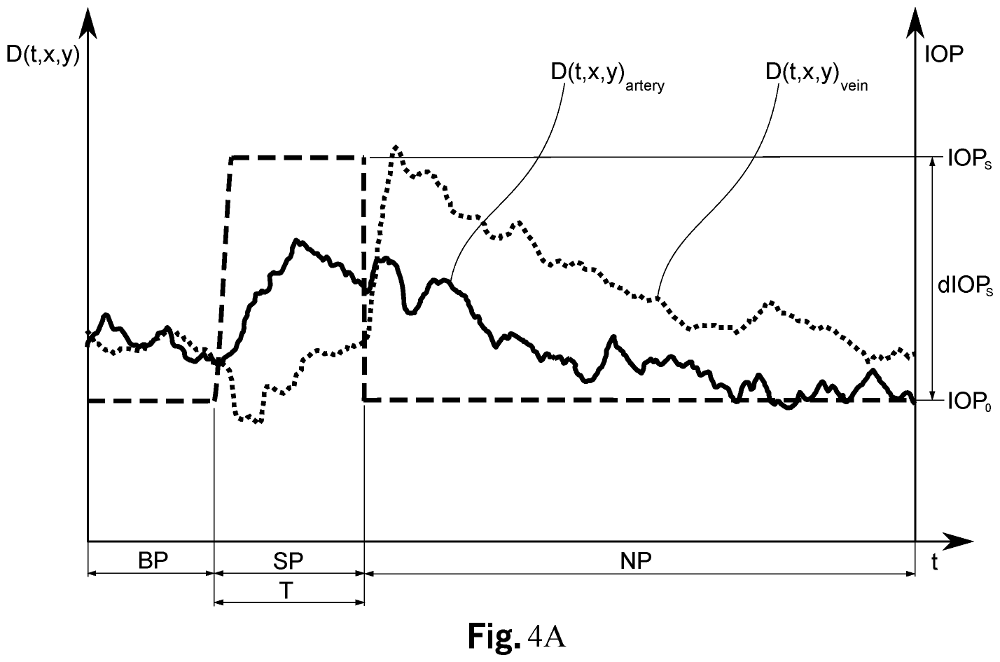

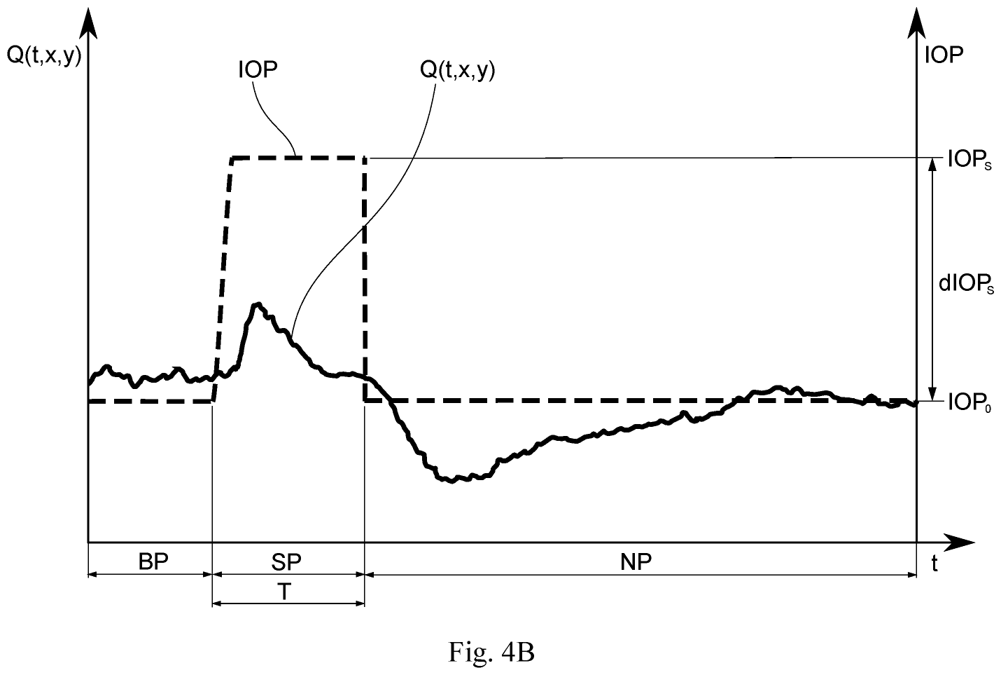

The recorded signals are printed out as a description for metabolic autoregulation, as e.g. diameter signals D(t,x,y) shown in FIG. 4A or quotient signals Q(t,x,y) shown in FIG. 4B, or characteristic measurement values, such as minima and maxima of the signals in the stimulation phase SP and from the posterior phase NP, are assigned to the measurement locations in a false color-coded manner and output as a mapping image superimposed on one of the video images of the retina (ocular fundus).

The reactions of the large vessels (arteries, veins) do not necessarily have to be examined together with the reactions of the capillaries, as in the exemplary embodiment described above. For certain medical questions, the examination of only one of the vessel types, i.e. arteries, capillaries or veins, is sufficient.

Furthermore, the method can also be performed semi-quantitatively by the examiner U manually determining the IOP values, in which case the corresponding longer pauses in the SD increase must be realized.

In a further embodiment of the invention, the individual correlation SD=f(IOP) could be determined separately from the stimulation in order to avoid possible tonographic effects and influences on vascular reactions.

### LIST OF REFERENCE NUMERALS

1 unit for generating and applying a stimulation pressure
1.1 pressure applicator
1.2 pressure generating unit
1.3 holder
2 imaging unit
3 tonometer
4 computing and control unit
5 input and output unit
7 results image storage unit
8 data and image evaluation unit
9 signal analysis unit
10 unit for generating spectrally normalized quotient signals
11 unit for generating vessel diameter signals
A eye
U examiner
rPP retinal perfusion pressure (value)
rPPₛ stimulation perfusion pressure
rPa retinal arterial blood pressure
rPv retinal venous blood pressure (within the eyeball)
RVP retinal venous blood pressure (value) outside the eyeball
SD stimulation pressure (value)
IOP intraocular pressure (value)
IOP₀ resting intraocular pressure (value)
dIOPₛ predetermined change intraocular pressure (value)
IOPₛ stimulation intraocular pressure value
SDₛ stimulation pressure value associated with the stimulation intraocular pressure value IOPₛ
BP baseline phase
SP stimulation phase
NP posterior phase
T stimulation period
Q(t,x,y) (spectrally normalized) quotient signal
D(t,x,y) vessel diameter signal

1. A device for examining metabolic autoregulation of retinal vessels in a patient's eye (A), said device comprising:
a unit for generating and applying stimulation pressure acting on the eye (A);
imaging unit being a modified retinal camera with a digital image sensor, the imaging unit serving to generate video sequence of images of retina, to each of the images being assigned two color channels;
a unit for generating spectrally normalized quotient signals which derives quotient signals (Q(t,x,y)) from intensity signals of the color channels, the quotient signals (Q(t,x,y)) being utilized to infer a vascular reaction and the metabolic autoregulation of the capillaries of the retinal vessels; or
imaging unit using laser scanning technology or optical coherence tomography for generating a video sequence of images of the retina derive images signals corresponding to local vessel diameters, local blood velocities, local blood flows or local capillary densities of the capillaries or/and the large vessels;
a tonometer for measuring an intraocular pressure (IOP) in the eye (A), said intraocular pressure (IOP) changing as a function of a stimulation pressure (SD) applied by the unit for generating and applying the stimulation pressure; and
a sensor for measuring the stimulation pressure (SD) comprised in the unit for generating and applying a stimulation pressure the sensor serving to assign a stimulation pressure value to a respective measured intraocular pressure value (IOP) and to each image of the video sequence.

2. The device according to claim 1, wherein the unit for generating and applying a stimulation pressure comprises a pressure applicator which can be attached to a patient's head, in a fixed manner with respect to the patient's eye (A) outside of a cornea and outside of a light path of the imaging unit in a pressure-free planar contact with the eye (A).

3. The device according to claim 1, wherein the imaging unit is the spectrally modified retinal camera having an illumination beam path, and wherein -a double band-pass filter with a spectral range in red light and a spectral range in green light is disposed in the illumination beam path.

4. A method for examining metabolic autoregulation of retinal vessels in a patient's eye (A), the method comprising:
recording a video sequence of images of the retinal vessels during a baseline phase (BP) which does not affect the eye (A);
recording the video sequence of images of the retinal vessels during a stimulation phase (SP), increasing an intraocular pressure (IOP) by a predetermined change intraocular pressure value (dIOPₛ) by applying and increasing a stimulation pressure (SD) acting on the eye (A), and maintaining the intraocular pressure (IO) at a stimulation intraocular pressure value (IOPₛ) for a stimulation period (T);
recording the video sequence of images of the retinal vessels during a posterior phase (NP) which does not affect the eye (A), and deriving signals corresponding to local vascular perfusion from the images of the video sequence;
carrying out the increase by the predetermined change intraocular pressure value (dIOPₛ) starting from a measured resting intraocular pressure value (IOP₀) if, during the baseline phase (BP), measurement criteria for a spontaneous venous collapse at an optic nerve head are determined on the retina;
carrying out the increase by the predetermined change intraocular pressure value (dIOPₛ) starting from an increased intraocular pressure value (IOP_{RVP}), if, during an increase in intraocular pressure (IOP), measurement criteria for spontaneous venous collapse at the optic nerve head are determined on the retina when the increased intraocular pressure value (IOP_{RVP}) is reached; and
carrying out the increase by the predetermined
change intraocular pressure value (dIOPₛ) on the basis of the measured resting intraocular pressure value (IOP₀) if no spontaneous venous collapse is detected, not even during the increase in intraocular pressure (IOP) at the optic nerve head on the retina.

5. The method according to claim 4, wherein the signals corresponding to the vascular reaction of the retinal vessels for arterial and venous vessels are vessel diameter signals (D(t,x,y)), and wherein the signals describing capillary vessels are spectrally normalized quotient signals (Q(t,x,y)) which are acquired and recorded in parallel.

6. The method according to claim 4, further comprising measuring at least a second intraocular pressure value after measuring the resting intraocular pressure value (IOP₀) the stimulation pressure (SD), and determining for the eye (A) an individual relationship between the intraocular pressure values (IOP) and the respectively associated stimulation pressure values for setting the stimulation intraocular pressure value (IOPₛ) via an individually associated stimulation pressure value (SDₛ).

7. The method according to claim 4, further comprising increasing the stimulation pressure (SD) by at least 1 mmHG per second with the start of the stimulation phase (SP) to rapidly increase the resting intraocular pressure value (IOP₀) by the predetermined change intraocular pressure value (dIOPₛ) to the stimulation intraocular pressure value (IOPₛ).

8. The method according to claim 4, further comprising terminating the stimulation phase (SP) after the stimulation period (T) has elapsed, and the stimulation pressure (SD) is reduced to zero.
